(19) United States
(12) Patent Application Publication
(10) Pub. No.: US 2009/0169548 A1
(43) Pub. Date: Jul. 2, 2009

(54) BINDING MOLECULES

(76) Inventors: Franklin Gerardus Grosveld, Rotterdam (NL); Richard Wilhelm Janssens, Rotterdam (NL); Drabek Dubravka, Rotterdam (NL); Roger Kingdon Craig, Cheshire (GB)

Correspondence Address: COZEN O'CONNOR, P.C., 1900 MARKET STREET, PHILADELPHIA, PA, 19103-3508, US

(21) Appl. No.: 12/161,986

(22) PCT Filed: Jan. 25, 2007
(86) PCT No.: PCT/GB07/00258

§ 371 Date: Jul. 24, 2008

(30) Foreign Application Priority Data
Jan. 25, 2006 (GB) 0601513.5

(51) Int. Cl. A61K 39/395; C07K 16/18; C12N 15/11; C12N 15/00; A61P 43/00; A61K 31/7088; C12N 5/06; C12P 21/04

(52) U.S. Cl. 424/133.1; 530/387.3; 536/23.53; 435/320.1; 435/358; 435/69.6; 514/44

(57) ABSTRACT

The present invention relates to the manufacture of mono, di and multivalent polypeptide binding complexes, also mono, di or multispecific polypeptide binding complexes and uses thereof. The invention also relates to the manufacture and use of a diverse repertoire of antigen specific VH binding domains derived from phage display libraries, transgenic animals or natural sources. Preferably the VH binding domains and the dimerisation domains comprise human sequences. The polypeptide binding complexes comprise homo or heterodimerisation domains with four antigen binding [VH] domains fused at the amino and carboxyl termini of the dimerisation domains preferably using natural hinge or linker peptides. Where the polypeptide binding complexes lack CH2-CH3 effector functions they are preferably less than 120 kDa in size. Routes of manufacture are described herein.

### FIELD OF THE INVENTION

The present invention relates to the generation of polypeptide binding complexes comprising VH binding domains (as defined herein) linked to both amino and carboxyl termini of dimerisation domains. VH binding domains and dimerisation domains generated using the methods of the present invention show inherent structural and functional stability relative to scFv derived polypeptide binding complexes described in the prior art, so providing advantages for product manufacture and product stability. The uses thereof are also described.

### BACKGROUND TO THE INVENTION

Monoclonal antibodies or variants thereof will represent a high proportion of new medicines launched in the 21^{st} century. Monoclonal antibody therapy is already accepted as a preferred route for the treatment for rheumatoid arthritis and Crohn's disease and there is impressive progress in the treatment of cancer. Antibody-based products are also in development for the treatment of cardiovascular and infectious diseases. Most marketed monoclonal antibody products recognise and bind a single, well-defined epitope on the target ligand (eg TNFα). The assembly of a complex consisting of two heavy chains and two light chains (the H₂L₂ complex) and subsequent post-translational glycosylation processes require the use of mammalian production systems. Production costs and capital costs for antibody manufacture by mammalian cell culture are high and threaten to limit the potential of antibody-based therapies in the absence of acceptable alternatives. A variety of transgenic organisms are capable of expressing fully functional antibodies. These include plants, insects, chickens, goats and cattle. Functional antibody fragments can be manufactured in E. coli but the product generally has low serum stability unless pegylated during the manufacturing process.

Bi-specific antibody complexes are engineered Ig-based molecules capable of binding two different epitopes on either the same or different antigens. Bi-specific binding proteins incorporating antibodies alone or in combination with other binding agents show promise for treatment modalities where captured human immune functions elicit a therapeutic effect, for example the elimination of pathogens (Van Spriel et al., (1999) J. Infect. Diseases, 179, 661-669; Tacken et al., (2004) J. Immunol., 172, 4934-4940; U.S. Pat. No. 5,487,890), the treatment of cancer (Glennie and van der Winkel, (2003) Drug Discovery Today, 8, 503-5100); and immunotherapy (Van Spriel et al., (2000) Immunol. Today, 21, 391-397; Segal et al., (2001) J. Immunol. Methods, 248, 1-6; Lyden et al., (2001) Nat. Med., 7, 1194-1201).

Manufacturing issues are compounded where a bi-specific antibody product is based on two or more H₂L₂ complexes. For example, co-expression of two or more sets of heavy and light chain genes can result in the formation of up to 10 different combinations, only one of which is the desired heterodimer (Suresh et al., (1986) Methods Enzymol., 121, 210-228).

To address this issue, a number of strategies have been developed for the production in mammalian cells of full length bi-specific IgG formats (BsIgG) which retain heavy chain effector function. BsIgGs require engineered “knob and hole” heavy chains to prevent heterodimer formation and utilise identical L-chains to avoid L-chain mispairing (Carter, (2001) J. Immunol. Methods, 248, 7-15). Alternative chemical cross-linking strategies have also been described for the production of complexes from antibody fragments each recognising different antigens (Ferguson et al., (1995) Arthritis and Rheumatism, 38, 190-200) or the cross-linking of other binding proteins, for example collectins, to antibody fragments (Tacken et al., (2004) J. Immunol., 172, 4934-4940).

The development of diabodies or mini antibodies (BsAb) generally lacking heavy chain effector functions also overcomes heterodimer redundancy. These comprise minimal single chain antibodies incorporating V_{H} and V_{L} binding sites (scFv) which subsequently fold and dimerise to form a divalent bi-specific antibody monovalent to each of their target antigens (Holliger et al., (1993) PNAS, 90, 6444-6448; Muller et al., (1998) FEBS Lett., 422, 259-264). In one instance, C_{H}1 and L-constant domains have been used as heterodimerisation domains for bi-specific mini-antibody formation (Muller et al., (1998) FEBS Lett., 259-264). A variety of recombinant methods based on E. coli expression systems have been developed for the production of BsAbs (Hudson, (1999) Curr. Opin. Immunol., 11, 548-557), though it would appear that the cost and scale of production of clinical grade multivalent antibody material remains the primary impediment to clinical development (Segal et al., (2001) J. Immunol. Methods, 248, 1-6).

Recently, the BsAb concept has been extended to encompass di-diabodies, tetravalent bi-specific antibodies where the V_{H} and V_{L} domains on each H and L chain have been replaced by engineered pairs of scFv binding domains. Such constructs, whilst complex to engineer, can be assembled in mammalian cells in culture in the absence of hetero-dimer redundancy (Lu et al., (2003) J. Immunol. Methods, 279, 219-232).

The structure of immunoglobulins is well known in the art. Most natural immunoglobulins comprise two heavy chains and two light chains. The heavy chains are joined to each other via disulphide bonds between hinge domains located approximately half way along each heavy chain. A light chain is associated with each heavy chain on the N-terminal side of the hinge domain. Each light chain is normally bound to its respective heavy chain by a disulphide bond close to the hinge domain.

When an Ig molecule is correctly folded, each chain folds into a number of distinct globular domains joined by more linear polypeptide sequences. For example, the light chain folds into a variable (V_{L}) and a constant (C_{L}) domain. Heavy chains have a single variable domain V_{H}, adjacent the variable domain of the light chain, a first constant domain, a hinge domain and two or three further constant domains. Interaction of the heavy (V_{H}) and light (V_{L}) chain variable domains results in the formation of an antigen binding region (Fv). Generally, both V_{H} and V_{L} are required for optimal antigen binding, although heavy chain dimers and amino-terminal fragments have been shown to retain activity in the absence of light chain (Jaton et al., (1968) Biochemistry, 7, 4185-4195).

With the advent of new molecular biology techniques, the presence of heavy chain-only antibody (devoid of light chain) was identified in B-cell proliferative disorders in man (Heavy Chain Disease) and in murine model systems. Analysis of heavy chain disease at the molecular level showed that mutations and deletions at the level of the genome could result in inappropriate expression of the heavy chain C_{H}1 domain, giving rise to the expression of heavy chain-only antibody lacking the ability to bind light chain (see Hendershot et al, (1987) J. Cell Biol., 104, 761-767; Brandt et al., (1984) Mol. Cell. Biol., 4, 1270-1277).

Separate studies on isolated human V_{H} domains derived from phage libraries (Ward et al., (1989) Nature, 341, 544-546) demonstrated antigen-specific binding of V_{H} domains but that these V_{H} domains generally but not always proved to be of relatively low solubility (see Jespers et al. (2004) J. Mol. Biol. 337, 893-903).

Studies using other vertebrate species have shown that camelids, as a result of natural gene mutations, produce functional IgG2 and IgG3 heavy chain-only dimers which are unable to bind light chain due to the absence of the C_{H}1 light chain-binding region (Hamers-Casterman et al., (1993) Nature, 363, 446-448) and that species such as shark produce a heavy chain-only-like binding protein family, probably related to the mammalian T-cell receptor or immunoglobulin light chain (Stanfield et al., (2004) Science, 305, 1770-1773).

A characterising feature of the camelid heavy chain-only antibody is the camelid V_{H} domain, which provides improved solubility and stability relative to the natural human V_{H} domain. Human V_{H} domains may be engineered for improved solubility characteristics (see Davies and Riechmann, (1996) Protein Eng., 9 (6), 531-537; Lutz and Muyldermans, (1999) J. Immuno. Methods, 231, 25-38) or solubility maybe be acquired by natural selection in vivo (see Tanha et al., (2001) J. Biol. Chem., 276, 24774-24780; Jespers L, Schon O, James L C, Veprintsev D, Winter G., J Mol. Biol. 2004 Apr. 2; 337(4):893-903). However, where V_{H} binding domains have been derived from phage libraries, intrinsic affinities for antigen remain in the low micromolar to high nanomolar range, in spite of the application of affinity improvement strategies involving, for example, affinity hot spot randomisation (Yau et al., (2005) J. Immunol. Methods, 297, 213-224).

scFvs, have limitations due to inherent instability and folding inefficiency when produced and recovered from host cells, or when produced as intrabodies in a reducing intracellular environment (see der Maur et al., (2002) J. Biol. Chem. 277, 45075-45085). In contrast V_{H} domains, typified by camelid V_{HH}, show high thermodynamic stability relative to conventional antibody fragments (Dumoulin et al., (2002) Protein Science, 11, 500-515) and retain functional stability even in the presence of non-ionic and anionic surfactants, and harsh denaturing conditions such as urea (Dolk et al., (2005) Applied and Environmental Microbiology, 71, 442-450), important features for the recovery of functional antibody complexes in high yield from harsh manufacturing environments, and the maintenance of product structural and functional integrity both in vivo and in vitro. V_{HH} and camelised or engineered V_{H} antibody domains also show the potential for greater target penetration of infectious agents than larger conventional antibody fragments (Stijlemans et al., (2004) J. Biol. Chem. 279, 1256-1261) and, when used as an “intrabody”, retain intracellular structural and functional stability, blocking the production of porcine retrovirus by PK15 cells in culture (Dekker et al., (2003) J. Virol. 77, 12132-12139). Camelid V_{H} antibodies are also characterised by a modified CDR3 loop. This CDR3 loop is, on average, longer than those found in non-camelid antibodies and is a feature considered to be a major influence on overall antigen affinity and specificity, which appear to compensate for the absence of a V_{L} domain in the camelid heavy chain-only antibody species (Desmyter et al., (1996) Nat. Struct. Biol., 3, 803-811, Riechmann and Muyldermans, (1999) J. Immunol. Methods, 23, 25-28).

Recently, methods for the production of heavy-chain-only antibodies in transgenic mammals have been developed (see WO02/085945 and WO02/085944). Functional heavy chain-only antibody of potentially any class (IgM, IgG, IgD, IgA or IgE) and derived from any mammal (including man) can be produced from transgenic mammals (preferably mice) as a result of antigen challenge.

Heavy chain-only monoclonal antibodies can be recovered from B-cells of the spleen by standard cloning technology or recovered from B-cell mRNA by phage display technology (Ward et al., (1989) Nature, 341, 544-546). Heavy chain-only antibodies derived from camelids or transgenic animals are of high affinity. Structural studies based on antibodies raised in transgenic mice as a result of antigen challenge shows that camelised human V_{H} antibody diversity is largely driven by in vivo maturation processes, with dependency on VDJ recombination events and somatic mutation. However, unlike the camelid V_{HH}, the CDR3 loop is absent from camelised human VH where the CDR3 region is derived from human D and J regions (see Janssen et al., (2006) PNAS 103 (41):15130-5. Epub 2006 Oct. 2* and PCT/GB2005/002892).

An important and common feature of the V_{H} domains found in heavy chain-only antibodies, such as camelid V_{HH} heavy chain-only antibodies and camelised human V_{H} heavy chain only antibodies, is that each region binds as a monomer with no dependency on dimerisation with a V_{L} region for optimal solubility and binding affinity. These features appear particularly suited to the production of blocking agents and tissue penetration agents (for review see Holliger, P. & Hudson, P. J. (2005) Nature Biotechnology 23, 1126-1136).

However, the benefits of V_{H} domains found in heavy chain-only antibodies have yet to be used to advantage in design of multimeric proteins as reagents, therapeutics and diagnostics, although two V_{H} domains tethered by a natural antibody hinge region have been shown to retain binding characteristics within bi-specific or bivalent constructs (Conrath et al., (2001) J. Biol. Chem. 276, 7346-7352).

The incorporation of multiple binding domains in combination with a dimerisation domain has clear advantage over parallel approaches using scFvs which must be engineered from V_{H} and V_{L} domains with the associated potential of loss of specificity and avidity, the increased risk of antigenicity due to the presence of linker peptides, and inherent lack of stability relative to V_{H} binding domains. V_{H} binding domains derived from antibody-related gene families such as T-cell receptors or the shark immunogloblin family also provide alternatives to scFv for the generation of bi- or multi-specific binding molecules.

The presence of heavy chain C_{H}2 and C_{H}3 constant domains provides the basis for the stable dimerisation seen in natural antibodies, and provides recognition sites for post-translational glycosylation in addition to heavy chain effector functions. C_{H}2-C_{H}3 dimerisation domains have been used in the design of tetrameric monospecific homodimers or bivalent bi-specific homodimers carrying scFv binding domains at their amino and carboxyl termini (see Jendreyko et al. (2003) J. Biol. Chem. 278, 47812-47819) or combinations of scFv binding domains and receptor binding proteins (Biburger et al. (2005) J. Mol. Biol. 346,1299-1311). C_{H}2-C_{H}3 domains have also been used to construct bivalent bi-specific homodimers using, camelised V_{H} and llama V_{HH} domains found in heavy chain-only antibodies (PCT/GB2005/002892).

There remains a need in the art to improve over available scFv binding technology and provide antigen-specific, soluble and structurally stable mono-valent, bi-valent or multi-valent polypeptide binding complexes. Dimerisation domains may comprise natural or engineered immunoglobulin C_{H}2-C_{H}3 dimerisation domains lacking heavy chain effector function for example C_{H}2-C_{H}3 derived from IgG4 (see Bruggemann, M. et al. J. Ex. Med. (1987) 166, 1351-1361). Preferably dimerisation domains other than C_{H}2-C_{H}3 are incorporated. Preferably, the resulting polypeptide binding complex is less than 120 kDa molecular weight so as to maximise tissue penetration when administered in vivo.

### BRIEF SUMMARY OF THE INVENTION

The present invention provides a method to use VH binding domains (as defined herein) alone or in combination with other binding domains, but excluding scFVs, to produce a polypeptide binding complex.

According to the invention there is provided a polypeptide binding complex comprising a dimer of a first heavy chain and a second heavy chain, wherein each heavy chain comprises an amino terminal VH binding domain (as defined herein); a carboxy terminal VH binding domain (as defined herein); and a dimerisation domain preferably lacking C_{H}2-C_{H}3 dimerisation functionality.

The term “VH binding domain” as used herein includes natural VH binding domains, for instance as expressed by a heavy chain locus alone as a result of recombination between single V, D and J gene segments followed subsequently by somatic mutation. The term “VH binding domain” encompasses any naturally occurring antigen-binding domain derived from a vertebrate, including shark, camelid and human. Where the VH binding domain is taken from a camelid or other natural heavy chain-only antibody, it is referred to as a V_{HH} domain. Where the VH domain is taken or derived from an antibody other than a heavy chain-only antibody, it is referred to as a V_{H} domain. “VH binding domain” includes V_{H} or V_{HH} domains which have been altered through selection or engineering to change their characteristics. For example, stability under certain conditions or solubility may have been altered. The VH domain may also have been altered through selection or engineering to more closely resemble a V_{H} or V_{HH} domain from another species. For example, a V region of a human V_{H} domain may have been altered to more closely resemble a V region found in a camelid V_{HH} domain. The term “VH binding domain” also includes homologues, derivatives or protein fragments, which are capable of functioning as a VH domain, for example a VL binding domain. All such embodiments are included in the invention.

Alternatively, the polypeptide binding complex may comprise a dimer of a first heavy chain and a second heavy chain, wherein each heavy chain comprises one or more additional amino terminal VH binding domains in tandem and separated by a hinge domain; and one or more additional carboxy terminal VH binding domains in tandem and separated by a hinge domain.

For therapeutic applications, the dimerisation domain is preferably of human origin, and may, depending on the application, comprise natural or engineered glycosylation sites to enhance plasma stability or alternatively may lack all post-translational modification sites to enhance plasma clearance or to reduce masking so as to enhance target recognition and binding. Where performance criteria in vivo, such as tissue penetration or plasma clearance, are required, the size of the overall polypeptide binding complex should preferably not be greater than 120 kDa.

Where the polypeptide binding complex comprising VH binding domains is to be used as an intrabody (see Dekker et al. (2003) J. Virol. 77, 12132-12139), additional, intracellular signalling features may be incorporated to determine, for example, intranuclear or membrane localisation (see, for example, Jendreyo et al., (2003) J. Biol. Chem. 278, 47812-47819). For manufacturing purposes, signal peptides may also be incorporated in vectors at the amino termini of polypeptide binding complexes so as to facilitate synthesis and secretion of the assembled polypeptide complex from production cells of choice (eg yeast, insect or mammalian cells). The dimerisation domain may comprise a homodimer or a heterodimer.

In one embodiment, the dimerisation domain of the first heavy chain is different to that of the second heavy chain, such that the polypeptide binding complex is a heterodimer comprising different polypeptides (heterodimers).

In an alternative embodiment, the dimerisation domain of the first heavy chain is the same as that of the second heavy chain, such that the polypeptide binding complex is a homodimer comprising two identical polypeptides (homodimers).

The VH domains of the invention may show the same specificity or they may show different specificity. Where the polypeptide binding complex comprises four VH domains, these may be tetravalent monospecific, bivalent bi-specific, trispecific or tetraspecific. Where there are more than four VH domains, then polypeptide binding complexes are envisaged which show greater increasing levels of specificity in line with the number of additional VH domains. For example, a polypeptide complex with eight VH domains may show octaspecificity.

Where rapid clearance or enhanced tissue penetration is required, then preferably, the polypeptide binding complex is less than 120 kDA in size.

In an alternative embodiment, one or more, but not all of the VH domains may be substituted by an alternative class of polypeptide binding domain. Preferably, the alternative binding domain is a cytokine, a growth factor, a receptor antagonist or agonist or a ligand.

Preferably, the dimerisation domain and/or the amino or carboxy terminal binding domains of one or both of the heavy chains are separated by a flexible hinge domain.

The invention also provides an isolated nucleic acid encoding the first heavy chain, second heavy chain or both heavy chains of the invention. The invention also provides a vector comprising the isolated nucleic acid. The invention further provides a cell transformed with the vector.

In another embodiment, the invention provides a method for the production of the polypeptide binding complex of the invention, comprising culturing a host cell transformed with a vector comprising a nucleic acid encoding the first heavy chain, second heavy chain or both heavy chains.

The VH binding domains, dimerisation domains or linker polypeptides of the invention may be produced by a synthetic route, such as peptide chemistry or chemical conjugation. The polypeptide binding complex may be pegylated to enhance stability in vivo.

The invention also provides a pharmaceutical composition comprising a polypeptide binding complex according to the invention. The invention also provides a method of treating a patient by administering a pharmaceutical composition or a vector of the invention to the patient.

The invention also provides the use of a polypeptide binding complex according to the invention in the preparation of a medicament for prophylaxis or treatment of disease.

The invention also provides using a polypeptide binding complex of the invention as a diagnostic, a reagent, an abzyme, an inhibitory agent, a cytochemical reagent, an imaging agent or an intrabody.

A polypeptide binding complex comprises a dimerisation domain configured with VH binding domains at both amino and carboxyl termini of the molecule. Optionally the dimerisation domain and the VH binding domain are separated by a flexible polypeptide linker. Preferred configurations comprise tetravalent monospecific polypeptide VH binding complexes and bivalent bi-specific polypeptide VH binding complexes (see FIGS. 1 to 5).

The VH binding domain maybe derived from any vertebrate though is preferably of human origin. Such VH binding domains maybe derived from natural sources such as camelid, transgenic animals or shark or selected from synthetic library arrays such as phage or yeast VH display libraries. VH binding domains maybe be engineered to improve physical characteristics, such as solubility and stability, or humanised to avoid or reduce antigenicity. The definition of VH encompasses any natural polypeptide binding domain derived from immunoglobulin heavy chain, immunoglobulin light chain, T-cell receptor or similar molecule, but excludes engineered scFv molecules where the binding site is engineered from the V_{H} and V_{L} domains of a tetrameric antibody (H₂L₂).

The dimerisation domain comprises a homodimer or heterodimer derived from a natural source, preferably human, which is stable under physiological conditions. The dimerisation domain may naturally incorporate addition effector functions or may be engineered to incorporate additional effector functions. These may include but are not limited to sites for post translational modifications (phosphorylation and glycosylation), sites for pegylation, enzymic, cytotoxic and imaging, immune stimulatory and receptor binding functions.

The present invention also provides a vector(s) comprising a nucleotide sequence encoding the VH polypeptide binding complex and dimerisation domain of the invention and a host cell transformed with such a vector(s).

Also provided is the use of a polypeptide binding complex according to the invention, in the preparation of a medicament. The polypeptide binding complexes of the invention may also be used as imaging agents, diagnostics, reagents, abzymes or inhibitory agents. Also provided is a pharmaceutical composition comprising the polypeptide binding complex according to the invention, and a pharmacologically appropriate carrier. The polypeptide binding complex of the invention may also be used as an intrabody whether delivered to the target cell as a vector capable of directing the intracellular synthesis of the polypeptide binding complex in the target cell, or delivered as a proteinaceous complex for cellular uptake and subsequent intracellular function within the target cell.

### DETAILED DESCRIPTION OF THE INVENTION

The present inventors have previously shown (see WO02/085945, WO02/085944 and PCT/GB2005/002892) that transgenic animals, in particular mice, can be generated using “micro loci” to produce class-specific VH heavy chain-only antibodies, or a mixture of different classes of VH heavy chain-only antibodies which are secreted by plasma or B cells in response to antigen challenge. These can then be used either to generate a reliable supply of class-specific, heavy chain-only antibody using established hybridoma technology or as a source of functional camelid V_{HH} binding domains or VH heavy chain-only binding domains, preferably soluble, V_{H} heavy chain-only binding domains of human origin. Similarly VH binding domains of the required specificity can be sourced from phage, yeast or similarly constructed display libraries.

Functional VH domains can be cloned and expressed in bacterial systems to generate VH binding domains with retention of antigen binding, specificity and affinity. Moreover VH binding domains retain functionality whether present at the amino or carboxyl terminus of a dimerisation domain. These features have been used to construct bivalent bi-specific homodimer VH binding molecules using the immunoglobulin heavy chain C_{H}2-C_{H}3 dimerisation region as a homo dimerisation domain (see PCT/GB2005/002892).

Taken together, these observations have important implications for the improved and simplified engineering of antibodies through the use of functionally stable, soluble VH domains. Tetravalent mono-specific VH binding complexes, or bivalent bi-specific VH binding complexes can be assembled using homo- or hetero-dimerisation domains and can be expressed and assembled using cells in culture (e.g. bacterial, yeast, insect, plant or mammalian cells) or by transgenic organisms (e.g. mammal, insect, plant etc) without the need for extensive prior engineering of the binding domain (scFv), the need for chemical cross linking or the need to separate the product from heterologous mixtures of mismatched binding domains.

VH domains are small (approx. 15 kDa) relative to scFv (28 kDa) or Fab (55 kDa) binding domains. Size differential and the presence or otherwise of heavy chain effector function has marked effect on the pharmacokinetics and biodistribution of protein complexes in vivo. Thus small soluble polypeptide binding complexes, which show rapid tissue penetration and high target retention, lack some or all effector functions and are rapidly cleared from the blood stream are superior in some clinical circumstances to large IgG molecules with poor tissue penetration, associated effector functions, and long serum half-lives (see Holliger, P. & Hudson, P. J. (2005) Nature Biotechnology, 23, 1126-1136 for extensive review). The use of most natural C_{H}2-C_{H}3 dimerisation domains adds heavy chain effector function to VH polypeptide binding complexes. The use of a C_{H}2-C_{H}3 domain derived from IgG4 or alternative homo- or hetero-dimerisation domains allows size constraints to be engineered in a controlled manner in the absence of heavy chain effector function, but with the incorporation of additional desired functional features as required.

The self association of proteins to form dimers and higher order oligomers through distinctive types of protein-protein interface requiring non-covalent interactions facilitates many biological functions (Ofran, Y.& Rost, B. (2003) J. Mol. Biol. 325, 377-387). Specific protein dimerisation is integral to biological function, structure and control (see Marianayagam et al. (2004) TIBS, 29, 618-625). Leucine zippers represent one well characterised structural motif capable of forming homo- and hetero-dimers (Landschulz et al., (1988) Science, 240, 1759-1764). C_{H}1 heavy chain domain and light chain constant domain form stable heterodimers. The carboxyl termini of certain eukaryotic transcriptional proteins, such as the TATA binding protein, form stable homodimers (Coleman et al., (1995) J. Biol. Chem. 270, 13842-13860). Numerous other dimerisation domains have been identified and characterised (see Brown, J. H. (2006) Protein Science 15, 1-13). Some, but not all, are suited to the development of polypeptide binding complexes. Preferred dimerisation domains are of human origin, preferably produced in specialised tissues so they are unlikely to be present as homologous contaminants during manufacture in diverse protein production systems. Alternatively, the dimerisation domain when present in the natural protein should have a nuclear or cytoplasmic location so endogenous protein can be segregated away from a dimerised polypeptide binding protein product destined for secretion via the secretory pathway using natural intracellular membrane bound processes. Preferably association/disassociation of the polypeptide dimer is not phosphorylation dependent.

VH polypeptide binding complexes, especially those of human origin, have wide ranging applications in the field of healthcare as medicines, imaging agents, diagnostics, abzymes and reagents, with parallel agricultural, environmental and industrial applications.

### Heavy Chain-Only Antibodies and Fragments Thereof

The antigen-specific VH binding domain of the invention may be cloned from, e.g., mRNA isolated from an antibody-producing cell of an immunised transgenic animal as described above. Cloned VH binding domain sequences can also be isolated from phage arrays (Ward et al., (1989) Nature, 341, 544-546) or similar array libraries, for example using yeast-based systems (Boder and Wittrup, (1997) Nat. Biotechnol., 15, 553-7). Antigen-specific VH binding domains can then be manufactured either alone or as fusion proteins in scalable bacterial, yeast or alternative expression systems. Sequences encoding VH binding domains can also be cloned from characterised hybridomas derived by classical procedures from immunised transgenic mice. These can then be used for the production of antigen specific VH binding domains and derivatives thereof.

Alternatively, VH domain-containing fragments can be generated from isolated immunoglobulin heavy chains, heavy chain-only antibodies derived from transgenic animals or natural sources (sharks and camelids) using enzymic or chemical cleavage technology and subsequent separation of the VH domain-containing fragment from the other cleavage products (Jaton et al., (1968) Biochemistry, 7, 4185-4195).

Where the VH binding domain is isolated from a characterised hybridoma, the VH binding domain sequence derived from mRNA can be directly cloned into an expression vector without recourse to additional selection steps necessary using phage and other display systems to characterise and optimise the affinity of the selected VH binding domain.

Production systems for VH binding domains incorporating heavy chain dimerisation and effector regions include mammalian cells in culture (e.g. B-cell hybridomas, CHO cells), plants (e.g. maize), transgenic goats, rabbits, cattle, sheep and chickens and insect larvae suited to mass rearing technology. Other production systems, including virus infection (eg baculovirus in insect larvae and cell-lines), are alternatives to cell culture and germline approaches. Other production methods will also be familiar to those skilled in the art. Suitable methods for the production of camelid heavy chain-only antibody or VH binding domains alone are known in the art. For example camelid VH binding domains have been produced in bacterial systems and camelid heavy chain-only homodimers have been produced in hybridomas and transfected mammalian cells (see Reichmann and Muyldermans, (1999) J. Immunol. Methods, 231, 25-38).

Methods are also well established for the expression of engineered human V_{H} binding domains derived using phage display technology (Tanha et al., (2001) J. Biol. Chem., 276, 24774-24780 and references therein).

Insect larvae from transgenic fly lines have been shown to produce functional heavy chain-only antibody fragments with characteristics indistinguishable from the same antibody produced by mammalian cells (PCT/GB2003/0003319).

The present invention also provides a vector(s) comprising a polypeptide binding protein, or fragment thereof, encoding VH binding domains and the dimerisation domain(s) according to the present invention.

The present invention also provides a host cell transformed with vectors according to the present invention.

In a first aspect, the present invention provides a polypeptide binding complex comprising an antigen-specific VH binding domains fused at carboxyl and amino terminal ends of a dimerisation domain lacking C_{H}2-C_{H}3 heavy chain effector function(s). These polypeptide binding complexes retain the physiological function conferred by the antigen-specific VH binding domain(s) in combination with additional targetting or effector functions naturally present or engineered into the dimerisation domain. Such polypeptide binding complexes may be in the form of functional mono-specific tetrameric binding complexes, bivalent bi-specific binding complexes, or tetra-specific binding complexes. VH binding domains are present at the amino and carboxy termini of the binding molecule (see FIG. 1 for example). Dimerisation domains may be homodimers or heterodimers dependent on the required design of the final functional polypeptide binding complex.

The advantages of this arrangement are several-fold. The presence of two or more identical VH domains acting in a co-operative manner provides a binding molecule of greater affinity and avidity than a single VH alone. Not only will the tetrameric VH product (e.g. a medicine) have greater potential potency than the monomeric or dimeric VH form, a tetravalent monospecific polypeptide binding complex assembled as a protein homodimer can be produced from a single cloned gene sequence as a single product free of mismatched contaminating binding sequences. A bivalent bi-specific polypeptide binding complex can facilitate cross-linking of different targets whilst retaining the beneficial cooperative effect of two VH binding domains for each antigen. For example, a bi-specific polypeptide complex may be utilised to enhance cell-cell interactions or cell/pathogen interactions. In this embodiment, the polypeptide complexes of the invention can be utilised, for example, to bridge between two cell types such as an erythrocyte and a pathogen (see Taylor et al., (1991) PNAS 88, 3305-3309). Bifunctionality can be used to simultaneously inhibit two components of an enzyme pathway (Jendreyko et al (2003) J. Biol. Chem. 278, 47812-47819).

Bifunctionality can be used to bring an effector moiety into close proximity with a target cell. Preferably, the VH binding domain at the amino terminal end of the each domain are identical and those at the carboxyl terminal end are identical (but recognise a different antigen or epitope to that at the amino terminal end), facilitating co-operative binding of pairs of VH binding domains.

The term ‘effector moiety’ as used herein includes any moiety that mediates a desired biological effect on a cell. The effector moiety is preferably soluble and may be a peptide, polypeptide or protein or may be a non-peptidic structure. For example, the effector moiety may be an enzyme, hormone, cytokine, drug, pro-drug, toxin, in particular a protein toxin, a radionuclide in a chelating structure, an imaging agent, albumin or an inhibitory agent. The effector moiety may be a cell, for example a T-cell, a peptide, polypeptide or protein or may be a non-peptidic structure. The effector moiety associated with the VH binding domain maybe cellular, proteinaceous, organic or inorganic in nature, dependent on the desired effect.

Albumin, immunoglobulins or other serum proteins may be utilised as an effector moiety to increase the stability or pharmacokinetic and/or pharmacodynamic properties of the antigen-specific VH binding domain (Sung et al., (2003) J. Interferon Cytokine Res., 23 (1): 25-36: Harmsen et al (2005) Vaccine, 23 (41) 4926-4934). Alternatively, the effector moiety may be a PEGylated structure or a naturally glycosylated structure so as to improve pharmacodynamic properties.

### Polypeptide Dimerisation Domains

The present inventors have also recognised the properties of any polypeptide binding complex are not just dependent on the VH binding domains incorporated in the final polypeptide binding complex. The size of the overall complex has significant influence on the pharmacokinetics of the complex in vivo and the ease of manufacture. Moreover, the dimerisation domain, dependent on the design of the polypeptide binding complex, may comprise additional effector activity. As such, in a second aspect of the invention, the polypeptide complex comprises a dimerisation domain which is limited in size so as to benefit tissue penetration. The second aspect of the present invention provides a dimerisation domain, wherein the dimerisation domain may comprise a homodimer, or a heterodimer. Preferably the dimerisation is through non-covalent interactions.

Dimerisation domains are linked covalently to VH binding domains at the dimerisation domains' amino and carboxyl termini.

Optionally, the polypeptide binding complex includes natural or engineered flexible hinge-like domains linking the VH binding domains and the dimerisation domain. The presence of hinge regions facilitates the independent function of the VH binding domains in the resultant polypeptide binding complex.

Optionally the dimerisation domain may comprise other useful functions, or may be engineered to incorporate additional features such as recognition sequences for glycosylation, pegylation, cell surface receptor binding, or tags for antibody or binding protein recognition. Dimerisation domains maybe engineered to optimise association through the introduction or elimination for example of additional cysteine residues.

Small dimerisation domains such as leucine zippers may be present as monomers or tandem pairs to enhance stability. Additional VH domains maybe used to link tandem dimerisation domains.

Preferably, the dimerisation domain has a size not greater than 60 kDa and the size of the polypeptide binding complex is approximately 120 kDA so as to enhance tissue penetration.

Preferred dimerisation domains comprise small domains from natural (human) proteins. These include the small 30 amino acid leucine zipper motifs present in many gene regulatory proteins (Landschulz et al. (1988) Science, 240, 1759-1764). This approach has been used previously for the production of bispecific F(ab′)₂ heterodimers (Kostelny et al., (1992) J. Immunology 148, 1547-1553). Zippers may be engineered to increase the specificity of a given heterodimerisation event (Loriaux et al., (1993) PNAS 90, 9046-9050).

A dimerisation domain according to the first and second aspect of the invention may be any protein, peptide fragment or consensus sequence capable of forming a homo or heterodimer protein-protein interaction, such as that seen between: C_{H}2-C_{H}3 region of the immunoglobulin heavy chain constant regions, the C_{H}1 domain of an immunoglobulin heavy chain and the constant region of an immunoglobulin light chain, or the homodimerisation of the 180 amino acid carboxyl terminal domain of the TATA binding protein (Colemen et al., (1995) J. Biol. Chem. 270, 13842-13849); VCAM and VLA-4; integrins and extracellular matrix proteins; integrins and cell surface molecules such as CD54 or CD102; ALCAMs; leucine zipper heterodimerisation domains; glutathione transfereases; and SRCR domains provide alternative examples.

Exemplary polypeptide binding complexes according to the first and second aspects of the invention are useful for cytochemical labelling, targetting methods or therapy. For example:
1. If an amino terminal antigen-specific VH binding domain targets a cancer cell surface marker, the carboxyl terminal VH may bind an effector moiety comprising a pro-drug converting enzyme. The amino terminal antigen-specific VH binding domain binds to the target and the carboxyl terminal VH brings the effector moiety into close proximity with the target such that the effector moiety can exert a biological effect on the target in the presence of the pro-drug (e.g. nitroreductase or DT diaphorase with CB1954);
2. If the amino and carboxyl terminal VH binding domains target a cytokine (e.g. TNFα), all four binding domains acting co-operatively will act with greater avidity and affinity than a VH monomer or dimer alone. Alternatively the amino terminal VH binding domains may bind a cytokine and the carboxyl domains serum albumin so as to enhance the serum half-life of the active complex.

The term ‘binding domain’ as used herein in respect of all the above aspects of the present invention includes any polypeptide binding domain that has effector activity in a physiological medium. Such a polypeptide binding domain must also have the ability to bind to a target under physiological conditions.

A VH binding domain may comprise a camelid VH domain or may comprise a VH domain obtained from a non-camelid. Preferably, the VH binding domain is a human VH binding domain. VH binding domains are preferably of B-cell origin, derived from transgenic animals or camelids (as described above) as opposed to VH domains derived from synthetic phage libraries, since the former will be of higher affinity due to their generation in response to antigen challenge in vivo via VDJ rearrangement and somatic mutation.

According to the third aspect of the invention some or all of the VH binding domains may be substituted by alternative protein binding domains. Preferably substitution occurs either at the amino or the carboxyl termini but not both.

Such binding domains include domains that can mediate binding or adhesion to a cell surface. Suitable domains which may be used in the polypeptide complexes of the invention are mammalian, prokaryotic and viral cell adhesion molecules, cytokines, growth factors, receptor antagonists or agonists, ligands, cell surface receptors, regulatory factors, structural proteins and peptides, serum proteins, secreted proteins, plasmalemma-associated proteins, viral antigens, bacterial antigens, protozoal antigens, parasitic antigens, lipoproteins, glycoproteins, hormones, neurotransmitters, clotting factors and the like, but excluding engineered single chain Fvs.

### Polynucleotide Sequences, Vectors and Host Cells

The present invention also provides a polynucleotide sequence encoding any one of the polypeptide binding complexes of the present invention, a vector comprising one or more of the polynucleotide sequences referred to above and a host cell transformed with a vector or vectors encoding the polypeptide binding complex of the present invention. The polynucleotides preferably include sequences which allow the expressed polypeptide binding complex to be secreted as either homo or heterodimers into the medium in which the host cell is growing. The host cell may include but is not limited to bacterial and yeast, insect, plant and mammalian host cells.

Furthermore, the present invention provides a transgenic organism expressing at least one homo- or hetero-dimer polypeptide binding complex of the present invention. The transgenic organism maybe a non-human vertebrate or mammal, a plant or an insect.

The production of polypeptide binding complexes for healthcare applications requires large scale manufacturing systems, examples of which are discussed in detail above. Such systems include plants (e.g. maize), transgenic cattle and sheep, and chickens, also insect larvae suitable for mass rearing technology. Other production systems, including virus infection (e.g. baculovirus in insect larvae and cell-lines) as an alternative to cell culture and germline approaches will also be familiar to those skilled in the art.

These methods, and other suitable methods known in the art, can be used for the production of the polypeptide binding complexes of the invention. Production of homodimers and/or of heterodimers can be achieved using these methods.

### Uses of Polypeptide Binding Complexes of the Invention

Polypeptide binding complexes of the invention have a great number of applications. For example, the polypeptide binding complexes of the invention comprise mono- bi- and multi-specific polypeptide complexes. These complexes are particularly advantageous, e.g. as therapeutics for the treatment, prevention and diagnosis of diseases. The polypeptide binding complexes of the invention are useful for cytochemical labelling, targeting methods, therapy and diagnostics.

In mono-antibody therapy, pathogen escape, for example due to a mutation leading to loss of a single binding site, will abolish the therapeutic effect of the antibody. The production of bivalent bi-specific polypeptide binding complexes recognising different antigens on the same pathogen can overcome this problem. The use of two VH binding domains having different specificities in the polypeptide binding complexes of the invention can also be utilised to enhance both cell-cell interactions and cell/pathogen interactions.

In this embodiment, the polypeptide complexes of the invention can be utilised, for example, to bridge polypeptide complexes between two cell types such as a pathogen and a macrophage, or a tumour cell and a T-cell. Alternatively the polypeptide complex may recognise two or more epitopes on the same pathogen with effector function being provided by receptor recognition domain within or inserted between the dimerisation domain and the hinge sequence.

Alternatively, bi-specific polypeptide binding complexes may be used to target cells and tissues in vivo, then subsequently to capture circulating effector molecules or imaging agents. For example bi-specific tumour targeting agents can be used to capture pro-drug converting complexes for the subsequent localised conversion of pro-drug to reactive agent. Bi- and multi-specific binding complexes in combination with effector agents may also be used to bind and destroy one or more pathogens dependent on the selection of binding domains. Alternatively, the presence of two or more binding domains which recognise different antigens on the same pathogen provide clinical advantages and reduce the likelihood of pathogen escape and drug redundancy as a result of mutation within the pathogen.

The first aspect of the present invention provides VH binding domains or fragments thereof and dimerisation domains including natural or engineered C_{H}2-C_{H}3 dimerisation domains lacking some or all heavy chain effector functions. According to the second aspect of the invention polypeptide binding complexes are not greater than 120 kDa in size so as to enhance tissue penetration of the polypeptide binding complex. According to the third aspect of the invention amino or carboxyl terminal VH binding domains maybe replaced with alternative binding domains excepting scFv. Polypeptide binding complexes comprising predominantly human sequences are suitable for pharmaceutical use in humans, and so the invention provides a pharmaceutical composition of the polypeptide binding complex comprising VH binding domains linked to a dimerisation domain through an optional hinge region at the amino and carboxyl termini. The invention also provides the use of a polypeptide binding complex of the present invention in the preparation of a medicament for the prophylaxis and/or treatment of disease. Where appropriate polypeptide binding complexes and effector moieties maybe formulated separately or together.

The pharmaceutical compositions and medicaments will typically be formulated before administration to patients.

For example, the polypeptide binding complexes may be mixed with stabilisers, particularly if they are to be lyophilised. Addition of sugars (e.g. mannitol, sucrose, or trehalose) is typical to give stability during lyophilisation, and a preferred stabiliser is mannitol. Human serum albumin (preferably recombinant) can also be added as a stabiliser. Mixtures of sugars can also be used, e.g. sucrose and mannitol, trehalose and mannitol, etc.

Buffer may be added to the composition, e.g. a Tris buffer, a histidine buffer, a glycine buffer or, preferably, a phosphate buffer (e.g. containing sodium dihydrogen phosphate and disodium hydrogen phosphate). Addition of buffer to give a pH between 7.2 and 7.8 is preferred, and in particular a pH of about 7.5.

For reconstitution after lyophilisation, sterile water for injection may be used. It is also possible to reconstitute a lyophilised cake with an aqueous composition comprising human serum albumin (preferably recombinant).

Generally, the polypeptide binding complexes will be utilised in purified form together with pharmacologically appropriate carriers.

The invention thus provides a method for treating a patient, comprising administering a pharmaceutical composition of the invention to the patient. The patient is preferably a human, and may be a child (e.g. a toddler or infant), a teenager or an adult, but will generally be an adult.

The invention also provides a polypeptide binding complex of the invention for use as a medicament.

The invention also provides the use of the polypeptide binding complexes of the invention in the manufacture of a medicament for treating a patient.

These uses, methods and medicaments are preferably for the treatment of one of the following diseases or disorders: wound healing, cell proliferative disorders, including neoplasm, melanoma, lung, colorectal, osteosarcoma, rectal, ovarian, sarcoma, cervical, oesophageal, breast, pancreas, bladder, head and neck and other solid tumours; myeloproliferative disorders, such as leukemia, non-Hodgkin lymphoma, leukopenia, thrombocytopenia, angiogenesis disorder, Kaposi's sarcoma; autoimmune/inflammatory disorders, including allergy, inflammatory bowel disease, arthritis, psoriasis and respiratory tract inflammation, asthma, immunodisorders and organ transplant rejection; cardiovascular and vascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection, pathological conditions associated with the placenta and other pathological conditions and for use in immunotherapy.

In a further aspect still, the present invention provides the use of a polypeptide binding complex of the present invention as a diagnostic, prognostic, or therapeutic imaging agent.

The present invention provides the use of a heavy chain-only antibody or a fragment thereof as herein described as an intracellular binding reagent, or an abzyme. Preferred heavy chain-only antibody fragments are soluble antigen-specific VH binding domains.

The present invention also provides, the use of VH polypeptide binding complexes according to the present invention as enzyme inhibitors or receptor blockers.

The present invention also provides the use of VH polypeptide binding complexes for use as a therapeutic, imaging agent, diagnostic, abzyme or reagent.

The present invention also provides VH polypeptide binding complexes for use as an intracellular binding agent (intrabody), and provides vectors functional in target cells for the intracellular expression of intrabodies. comprising VH polypeptide binding complexes.

### BRIEF DESCRIPTION OF THE DRAWINGS

FIG. 1: shows a polypeptide binding complex comprising a VH polypeptide binding domain, homo dimerisation domains linked by hinge or linker sequences. VH polypeptide domains are positioned at the amino and carboxy terminal ends of the dimerization domains.
A. Shows a tetravalent monospecific polypeptide binding domain
B. Shows a bivalent bi-specific polypeptide binding domain
C. Shows a monovalent tetraspecific polypeptide binding domain

FIG. 2: shows different configurations for heterodimerisation binding domains.

FIG. 3: shows a strategy for the generation of a tetravalent monospecific polypeptide binding complex

FIG. 4: shows a strategy for the generation of a bi-specific bivalent polypeptide binding complex with binding affinity for GAG and HSP

FIG. 5: shows an example of a bispecific tetravalent antibody comprising more than one amino and carboxy terminal VH domain.

FIG. 6: shows a scheme for generating heterodimerised bi-specific bi-valent binding molecules using fos and jun zipper domains.

FIG. 7: PCR results

### GENERAL TECHNIQUES

Unless defined otherwise, all technical and scientific terms used herein have the same meaning as commonly understood by one of ordinary skill in the art (e.g. in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2nd Ed. (1989) Cold Spring Harbor Laboratory Press, Cold Spring Harbor, N.Y. and Ausubel et al., Short Protocols in Molecular Biology (1999) 4th Ed., John Wiley & Sons, Inc.) and chemical methods. In addition Harlow & Lane, A Laboratory Manual, Cold Spring Harbor, N.Y., is referred to for standard Immunological Techniques.

Any suitable recombinant DNA technique may be used in the production of the bi- and multi-valent polypeptide complexes, single heavy chain antibodies, and fragments thereof, of the present invention. Typical expression vectors, such as plasmids, are constructed comprising DNA sequences coding for each of the chains of the polypeptide complex or antibody. Any suitable established techniques for enzymic and chemical fragmentation of immunoglobulins and separation of resultant fragments may be used. The identification, isolation and characterisation of antigen specific VH polypeptide binding domains from phage display libraries and hybridomas derived from camelids and transgenic mice use well established methodologies.

The present invention also provides vectors including constructs for the construction and expression of polypeptide binding complexes of the present invention.

It will be appreciated that a single vector may be constructed which contains the DNA sequences coding for more than polypeptide chain. For instance, the DNA sequences encoding two different polypeptide chains of a heterodimer with associated VH binding domains, may be inserted at different positions on the same plasmid.

Alternatively, the DNA sequence coding for each polypeptide chain, may be inserted individually into a plasmid, thus producing a number of constructed plasmids, each coding for a particular polypeptide chain. Preferably, the plasmids into which the sequences are inserted are compatible.

Each plasmid is then used to transform a host cell so that each host cell contains DNA sequences coding for each of the polypeptide chains in the polypeptide binding complex.

Suitable expression vectors which may be used for cloning in bacterial systems include plasmids, such as Col E1, pcR1, pBR322, pACYC 184 and RP4, phage DNA or derivatives of any of these.

For use in cloning in yeast systems, suitable expression vectors include plasmids based on a 2 micron origin.

Any plasmid containing an appropriate mammalian gene promoter sequence may be used in cloning in mammalian systems. Insect or bacculoviral promoter sequences may be used fir insect cell gene expression. Such vectors include plasmids derived from, for instance, pBR322, bovine papilloma virus, retroviruses, DNA viruses and vaccinia viruses.

Suitable host cells which may be used for expression of the polypeptide complex or antibody include bacteria, yeasts and eukaryotic cells, such as insect or mammalian cell lines, transgenic plants, insects, mammalian and other invertebrate or vertebrate expression systems.

### Polypeptide Binding Complexes of the Present Invention

It will be understood that term ‘polypeptide binding complex’, include homologous polypeptide and nucleic acid sequences obtained from any source, for example related cellular homologues, homologues from other species and variants or derivatives thereof. Thus, the present invention encompasses variants, homologues or derivatives of the polypeptide binding-complexes, VH binding domains and dimerisation domains as herein described.

In the context of the present invention, a homologous sequence is taken to include an amino acid sequence which is at least 80, 85, 90, 95, 96, 97, 98, 99, 99.5, 99.6, 99.7, 99.8, 99.9% identical, preferably at least 98 or 99%, identical, at the amino acid level over at least 30, preferably 50, 70, 90 or 100 amino acids. Although homology can also be considered in terms of similarity (i.e. amino acid residues having similar chemical properties/functions), in the context of the present invention it is preferred to express homology in terms of sequence identity.

The present invention also includes constructed expression vectors and transformed host cells for use in producing the polypeptide binding complexes, dimerisation domains and VH binding domains of the present invention.

After expression of the individual chains in the same host cell, they may be recovered to provide the complete polypeptide binding complex or VH in active form.

It is envisaged that, in preferred forms of the invention, the individual polypeptide binding complexes will be processed by the host cell to form the dimerised polypeptide binding complex which advantageously is secreted therefrom.

Techniques for the preparation of recombinant antibody polypeptide binding complexes is described in the above references and also in, for example, EP-A-0 623 679; EP-A-0 368 684 and EP-A-0 436 597.

### Uses of the Polypeptide Binding Complexes of the Present Invention

The polypeptide binding complexes including fragments thereof of the present invention may be employed in: in vivo therapeutic and prophylactic applications, in vitro and in vivo diagnostic applications, in vitro assay and reagent applications, and the like.

Therapeutic and prophylactic uses of the polypeptide binding complexes of the invention involve the administration of the above to a recipient mammal, such as a human.

Substantially pure polypeptide binding complexes including fragments thereof of at least 90 to 95% homogeneity are preferred for administration to a mammal, and 98 to 99% or more homogeneity is most preferred for pharmaceutical uses, especially when the mammal is a human. Once purified, partially or to homogeneity as desired, the polypeptide binding complexes as herein described may be used diagnostically or therapeutically (including extracorporeally) or in developing and performing assay procedures using methods known to those skilled in the art.

Generally, the polypeptide binding complexes of the present invention will be utilised in purified form together with pharmacologically appropriate carriers. Typically, these carriers include aqueous or alcoholic/aqueous solutions, emulsions or suspensions, which may include saline and/or buffered media. Parenteral vehicles include sodium chloride solution, Ringer's dextrose, dextrose and sodium chloride and lactated Ringer's.

Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The polypeptide complexes and antibodies, including fragments thereof, of the present invention may be used as separately administered compositions or in conjunction with other agents. These can include various immunotherapeutic drugs, such as cyclosporine, methotrexate, adriamycin, cisplatinum or an immunotoxin. Alternatively, the polypeptide binding complexes can be used in conjunction with enzymes for the conversion of pro-drugs at their site of action.

Pharmaceutical compositions can include “cocktails” of various cytotoxic or other agents in conjunction with the selected polypeptide binding complexes of the present invention or even combinations of the selected polypeptide binding complexes of the present invention.

The route of administration of pharmaceutical compositions of the invention may be any of those commonly known to those of ordinary skill in the art. For therapy, including without limitation immunotherapy, the polypeptide binding complexes of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counter-indications and other parameters to be taken into account by the clinician.

The polypeptide binding complexes and antibodies of this invention can be lyophilised for storage and reconstituted in a suitable carrier prior to use. Known lyophilisation and reconstitution techniques can be employed. It will be appreciated by those skilled in the art that lyophilisation and reconstitution can lead to varying degrees of functional activity loss and that use levels may have to be adjusted upward to compensate.

When used as an intrabody the polypeptide binding complex may be delivered using non-viral or viral based vectors, or maybe delivered as a liposomal or alternative formulation resulting in the uptake in the required target cell.

In addition, the polypeptide binding complexes of the present invention may be used for diagnostic purposes. For example, VH binding domains as herein described may be generated or raised against antigens which are specifically expressed during disease states or whose levels change during a given disease state. For diagnostic or reagent purposes polypeptide binding complexes can comprise VH domains binding one or more epitopes on the same antigen, alternatively one or more binding domains may act as capture domains either binding the polypeptide complex to a defined substrate or binding an assay component required for qualitative or quantitative aspects of the assay read out.

For certain purposes, such as diagnostic or tracing purposes, labels may be added. Suitable labels include, but are not limited to, any of the following: radioactive labels, NMR spin labels and fluorescent labels. Means for the detection of the labels will be familiar to those skilled in the art.

The compositions containing the polypeptide binding complexes of the present invention or a cocktail thereof can be administered for prophylactic and/or therapeutic treatments.

A composition containing one or more polypeptide binding complexes of the present invention may be utilised in prophylactic and therapeutic settings to aid in the alteration, inactivation, killing or removal of a select target cell population in a mammal. In addition, the selected repertoires of polypeptide binding complexes described herein may be used extracorporeally or in vitro selectively to kill, deplete or otherwise effectively remove a target cell population from a heterogeneous collection of cells.

### EXAMPLES

### Example 1

### Tetravalent Monospecific Anti-aTNF Polypeptide Binding Protein

The construct was derived from a previously characterised monoclonal antibody producing a heavy chain only IgM in a transgenic mouse challenged with aTNF. The VH domain comprised a camelid V segment and human DJ and constant regions.

The CH2CH3 backbone of the antibody was deleted and replaced by the CH1 immunoglobulin heavy chain domain and by the immunoglobulin light chain constant region. The VH domain was then duplicated and cloned at the carboxyl terminal end of each construct using a modified hinge region. This hinge was similar to the existing IgG2 hinge sequence but was altered by replacing the cysteines with prolines to prevent crosslinking of the cysteines in the antibody dimer and providing extra flexibility via the prolines to prevent the second antibody being spatially constrained, which otherwise may have inhibited its function.

Thus formation of the sulphide bridges normally present in the human IgG2 hinge, was prevented by replacing the cysteins (greyshade) with prolines (underlined). The prolines add extra flexibility to the hinge to allow the proper functioning of the second antibody domain that becomes connected to COOH terminus of the dimerisation domain via the hinge.

The normal IgG hinge sequence (cysteine codons in greyshade, proline codons underlined)

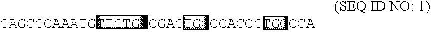

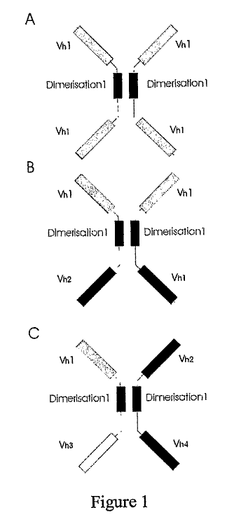

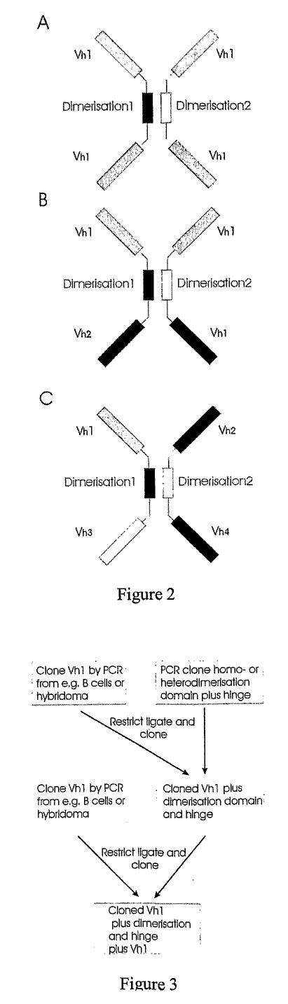

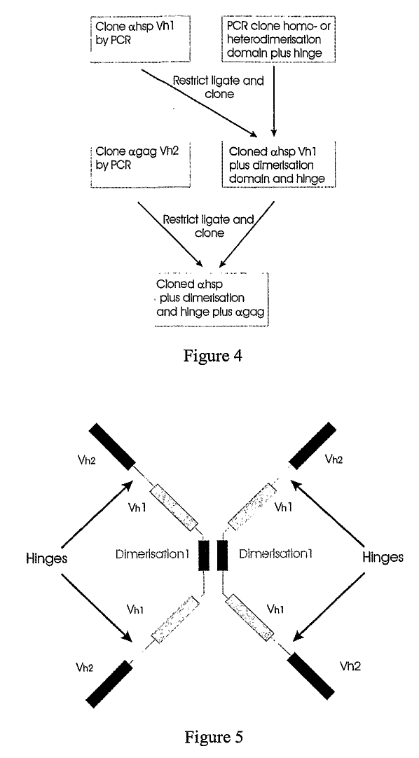

and its complement were replaced by AGCTTCTGAGCGCAAACCACCAGTCGAGCCACCACCGCCACCAC (SEQ ID NO:2) and its complement TCGAGTGGTGGCGGTGGTGGCTCGACTGGTGGTTTGCGCTCAGA (SEQ ID NO:3). This also provided the fragment (white box hinge, FIG. 2, center) with two single strand ends compatible with HindIII (bold) and XhoI (italic) sites for cloning purposes.

The final construct was ligated into a bluescript (Pbluescript11 sk+) expression plasmid that contains a chicken actin promoter and a CMV enhancer sequence (FIG. 22, expression plasmid) by standard recombinant DNA technology.

The diabody expression plasmids were grown and cotransfected with the plasmid pGK-hygro (to allow the selection of transfected cells) by standard methods (Superfect) into CHO cells. Positive clones were selected in hygromycin containing medium and positively identified as expressing the diabody by performing a standard aTNF ELISA of the growth medium containing secreted diabody by the CHO cells. Western blots of these ELISA selected clones under non-reducing and reducing conditions were performed in order to show that the protein expressed from the plasmid was a dimer compared to the monomer Thus the ELISA and the Western blot together show that the diabody is expressed and secreted into the medium as a dimer by the transfected CHO cells and that the antibody can bind aTNF. Comparison of the binding affinity of the aTNF VH monomer, -dimer and -tetramer showed maximum binding affinity with the tetramer.

### Example 2

### A Bi-Specific Bi-Valent Polypeptide Binding Complex Comprising VH Binding Domains and a CH2CH3 Dimerisation Domain Lacking Heavy Chain Effector Functions Derived from IgG4

The experiment was carried out using a camelised human VH domains raised against E. coli HSP70 protein at the amino terminus of the dimerisation domain, and a llama VHH domain raised against the PERV gag antigen (Dekker et al., (2003) J. Virol. 77, (22) 12132-9) at the carboxyl terminus. Experimental detail is as described in Example 2 FIGS. 22, 23 and 24 of PCT/GB2005/002892) except that the IgG2 CH2-CH3 dimerisation domain was replaced by a human IgG4 CH2-CH3 dimerisation domain (Bruggemann, M. et al. (1987) J. Ex. Med., 166, 1351-1361.

The vector comprising polypeptide binding complex was expressed in CHO cells, and the secreted polypeptide binding complex shown by western blotting to bind both HSP70 and gag antigens.

### Examples 3-5

Instead of using immunoglobulin constant regions other dimerisation domains can be used to generate multivalent multispecific bonding molecules, for example the leucine zipper domains of the jun and fos genes in combination with different (human) VH domains. The jun zipper domain can heterodimerise with the fos zipper domain, but it can also homodimerise. The following two examples describe the hetero- and homodimerisation using these zipper domains. The last example describes the use of other domains.

### Example 3

### Heterodimerised-Bi-Specific Bi-Valent Binding Molecules Using fos and jun Zipper Domains

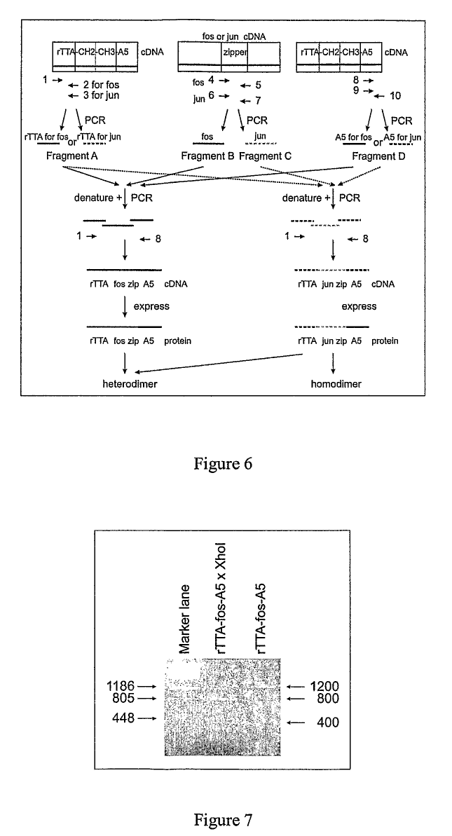

The basic scheme for the generation of such molecules is illustrated in FIG. 6 and consists of the following steps:
1. The VH developed against rTTA (Janssens et al., 2006) is amplified by PCR with primers that at the 5′ side has an EcoRI site (primer 1) and is homologous to the leader sequence and at the 3′ side is homologous to the hinge region plus a sequence homologous to the 5′ end of the fos and jun sequences (primer 2 and 3 respectively). Standard PCR amplification results in a fragment A for fos (FIG. 6 solid line) or fos (FIG. 7 dotted line) of 600 basepairs.

Primer 1:

(SEQ ID NO:4)

CTGGAATTCTCACCATGGAGCTGGGGCTGAGC
Primer 2:

SEQ ID NO:5)

CGCTTGGAGTGTATCAGTCAGTGGGCACCTGGGCACGGGGG
Primer 3:

(SEQ ID NO:6)

CAGCCGGGCGATTCTCTCCAGTGGGCACCTTGGGCACGGGGG

2. The fos and jun leucine zipper regions are amplified from human cDNA coding for fos or jun. The primers at the 5′ end contained a sequence homologous to the 3′ end of the hinge region of the rTTA VH (primers 4 and 5 respectively). The primers at the 3′ end were homologous to the 3′ end of the zipper regions (primers 6 and 7) and contains at the 3′ end a sequence homologous to the 5′ end of the hinge region (PCT/GB2005/002892) present at the 5′ end of the A5 VH (Janssens et al., 2006). Amplification of the fos and jun sequences results in fragments B (FIG. 6 solid line) and C (FIG. 6 dotted line) respectively of 200 bp each.

Primer 4:

(SEQ ID NO:7)

CCCCCGTGCCCAAGGTGCCCACTGACTGATACACTCCAAGCG
Primer 5:

(SEQ ID NO:8)

CCCCCGTGCCCAAGGTGCCCACTGGAGAGAATCGCCCGGCTG
Primer 6:

(SEQ ID NO:9)

TGGTGGTTTGCGCTCAGAAGCCAGGATGAACTCTAGTTTTTC
Primer 7:

(SEQ ID NO:10)

TGGTGGTTTGCGCTCAGAAGCAACGTGGTTCATGACTTTCTG

3. A hinge sequence not containing any cysteins is first cloned (as specified in PCT/GB2005/002892, ERKPPVEPPPPP) onto the A5 VH region (Dekker et al., 2003; Janssens et al., 2006). The hinge and A5 VH are subsequently amplified using a primer that is homologous with the 5′ end of the hinge region and a primer homologous to the 3′ end of the A5 VH region including the stop codon (primer, resulting in a fragment D for fos (FIG. 6 solid line) or jun (FIG. 6 dotted line) of 400 basepairs.

Primer 8:

(SEQ ID NO:11)

GAAAAACTAGAGTTCATCCTGGCTTCTGAGCGCAAACCACCA
Primer 9:

(SEQ ID NO:12)

CAGAAAGTCATGAACCACGTTGCTTCTGAGCGCAAACCACCA
Primer 10:

(SEQ ID NO:13)

GTCGAATTCTCATTCCGAGGAGACGGTGACCTGGGTC

4. Fragment A (FIG. 6 solid line), B and D (FIG. 6 solid line) are mixed in equimolar amounts and fragments A (FIG. 6 dotted line), C and D (FIG. 6 dotted line) are mixed in equimolar amounts, denatured and PCR amplified using the primers 1 and 10, resulting in a fragment of 1200 basepairs (see FIG. 7, rTTA-fos-A5 below, containing a characteristic XhoI site at the 5′ of the A5 sequence).
5. The rTTA-foszip-A5 and rTTA-junzip-A5 are cloned by standard means into a yeast (Pichia, Invitrogen) or standard CHO (between CAG promoter and polyA site) expression vector. These construct are introduced into yeast and CHO cells respectively.
6. Media and cells are collected and analysed by ELISA to show they still bind rTTA and A5 and native Western blots to show they are dimerised.

### Example 4

This is to show homodimerisation by the same method as shown in Example 3 with the exception that only the jun zipper part of the experiment is carried out. The rTTA-junzip-A5 is expressed in Pichia or CHO cells and shown to form rTTA and A5 binding homodimers by the same methods as in example 2.

### Example 5

Similar methodology as described in examples 3 and 4 can be applied for other homo- or heterodimer forming domains. For such cases the primers used in example 2, step 2 would be homologous to such other dimerising domains and the oligonucleotides used in steps 1 and 3 would have ends overlapping with these domains to enable step 4.

In all examples other VH or VL domains or other binding domains such as transcription factor DNA binding domains or ligand binding domains could be used.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology and biotechnology or related fields are intended to be within the scope of the following claims.

1. A polypeptide binding complex comprising a dimer of a first polypeptide chain and a second polypeptide chain, wherein each polypeptide chain comprises an amino terminal VH binding domain; a carboxy terminal VH binding domain; and a dimerisation domain wherein the dimerisation domain lacks C_{H}2-C_{H}3 functionality.

2. The polypeptide binding complex of claim 1 wherein the dimerisation domain is neither an engineered or natural CH2-CH3 domain.

3. The polypeptide binding complex of claim 1 or claim 2, wherein the dimerisation domain of the first polypeptide chain is different to that of the second polypeptide chain, such that the polypeptide binding complex is a heterodimer.

4. The polypeptide binding complex of claims 1 and 2, wherein the dimerisation domain of the first polypeptide chain is the same as that of the second polypeptide chain, such that the polypeptide binding complex is a homodimer.

5. The polypeptide binding complex of claim 1 or 2, wherein the four VH binding domains show the same specificity (tetravalent monospecific).

6. The polypeptide binding complex of claim 1 or 2, wherein the amino terminal VH binding domains show the same specificity; the carboxy terminal VH binding domains show the same specificity; and the binding specificity of the amino terminal and carboxy terminal VH domains differ (bivalent bi-specific).

7. The polypeptide binding complex of claim 1 or 2, wherein the amino terminal VH binding domains show the same specificity; and the carboxy terminal VH binding domains show different specificity to each other and to the amino terminal VH domains trispecific).

8. The polypeptide binding complex of claim 1 or 2, wherein the carboxy-terminal VH binding domains show the same specificity; and the amino terminal VH binding domains show different specificity to each other and to the carboxy terminal VH domains (trispecific).

9. The polypeptide binding complex of claim 1 or 2, wherein the amino terminal VH binding domains show different specificity to each other; and the carboxy terminal VH binding domains show different specificity to each other and to the amino terminal VH domains (tetraspecific).

10. The polypeptide binding complex of claim 1 or 2 not greater than 120 kDA in size.

11. The polypeptide binding complex of claim 1 or 2, wherein one or more of the VH binding domains maybe substituted by an alternative class of polypeptide binding domain.

12. The polypeptide binding complex of claim 1 or 2, wherein either the first polypeptide chain, the second polypeptide chain or both polypeptide chains further comprise a flexible hinge domain between either the amino terminal binding domain and the dimerisation domain; the carboxy terminal binding domain and the dimerisation; or both.

13. The polypeptide binding complex of claim 11, wherein the alternative binding domain is a cytokine, a growth factor, a receptor antagonist or agonist or a ligand.

14. The polypeptide binding complex according to claim 1 or 2, wherein each polypeptide chain further comprises one or more additional amino terminal VH binding domains in tandem and separated by a hinge domain; and one or more additional carboxy terminal VH binding domains in tandem and separated by a hinge domain.

15. An isolated polynucleotide encoding the first polypeptide chain, the second polypeptide chain or both polypeptide chains according to claim 1 or 2.

16. An expression vector containing the isolated polynucleotide of claim 15.

17. A host cell transformed with an expression vector of claim 16.

18. A method for the production of the polypeptide binding complex of claims claim 1 or 2, comprising culturing a host cell transformed with an expression vector containing an isolated polynucleotide encoding the first polypeptide chain, the second polypeptide chain, or both polypeptide chains of claim 1 or 2 and isolating the polypeptide complex.

19. A method producing a polypeptide binding complex of claim 1 or 2 which comprises:
transforming a host cell with a vector or vectors encoding a polypeptide binding complex of any one of claims 1 to 2:
growing the host cell under conditions which allow for the expression of the coding sequence(s) of the vector or vectors; and
harvesting the polypeptide binding complex from the host cell.

20. A method for the production of the polypeptide binding complex of claim 1 or 2, wherein the VH binding domain, dimerisation domain or linker polypeptides are produced by a synthetic route, such as peptide chemistry or conjugation.

21. A pharmaceutical composition comprising a polypeptide binding complex produced according to any one of claims 1 to 2.

22. The use of a polypeptide binding complex according to any one of claims 1 to 2 in the preparation of a medicament for prophylaxis or treatment of disease.

23. A method of treating a patient, comprising a pharmaceutical composition according to claim 22 to a patient in need of treatment.

24. The use of a polypeptide binding complex according to any one of claims 1 to 2 as a diagnostic, a reagent, an abzyme, an inhibitory agent, a cytochemical reagent or an imaging agent.

25. The use of a polypeptide binding complex according to any one of claims 1 to 2 as an intrabody.

26. A method of treating a patient, comprising administering a vector according to claim 16 to a patient in need of treatment.

27. A method of treating a patient, comprising administering a pharmaceutical composition according to claim 21 to a patient in need of treatment.


SEQUENCE LISTING (13 sequences)

SEQ ID NO: 1 - DNA, 36 bp, Artificial Sequence
  IgG hinge coding sequence
gagcgcaaatgttgtgtcgagtgcccaccgtgccca

SEQ ID NO: 2 - DNA, 44 bp, Artificial Sequence
  IgG hinge coding sequence replacement
agcttctgagcgcaaaccaccagtcgagccaccaccgccaccac

SEQ ID NO: 3 - DNA, 44 bp, Artificial Sequence
  IgG hinge coding sequence replacement complement
tcgagtggtggcggtggtggctcgactggtggtttgcgctcaga

SEQ ID NO: 4 - DNA, 32 bp, Artificial Sequence
  PCR Primer
ctggaattctcaccatggagctggggctgagc

SEQ ID NO: 5 - DNA, 42 bp, Artificial Sequence
  PCR Primer
cgcttggagtgtatcagtcagtgggcaccttgggcacggggg

SEQ ID NO: 6 - DNA, 42 bp, Artificial Sequence
  PCR Primer
cagccgggcgattctctccagtgggcaccttgggcacggggg

SEQ ID NO: 7 - DNA, 42 bp, Artificial Sequence
  PCR Primer
cccccgtgcccaaggtgcccactgactgatacactccaagcg

SEQ ID NO: 8 - DNA, 42 bp, Artificial Sequence
  PCR Primer
cccccgtgcccaaggtgcccactggagagaatcgcccggctg

SEQ ID NO: 9 - DNA, 42 bp, Artificial Sequence
  PCR Primer
tggtggtttgcgctcagaagccaggatgaactctagtttttc

SEQ ID NO: 10 - DNA, 42 bp, Artificial Sequence
  PCR Primer
tggtggtttgcgctcagaagcaacgtggttcatgactttctg

SEQ ID NO: 11 - DNA, 42 bp, Artificial Sequence
  PCR Primer
gaaaaactagagttcatcctggcttctgagcgcaaaccacca

SEQ ID NO: 12 - DNA, 42 bp, Artificial Sequence
  PCR Primer
cagaaagtcatgaaccacgttgcttctgagcgcaaaccacca

SEQ ID NO: 13 - DNA, 37 bp, Artificial Sequence
  PCR Primer
gtcgaattctcattccgaggagacggtgacctgggtc